(19) United States
(12) Patent Application Publication
(10) Pub. No.: US 2003/0150041 A1
(43) Pub. Date: Aug. 14, 2003

(54) Chemical-resistant, user-wearable protective article including a ceramic-based barrier layer

(76) Inventors: Hasmukh Patel, Maple Grove, MN (US); William E. Archibald, Maple Grove, MN (US)

Correspondence Address: GENERAL MILLS, INC., P.O. BOX 1113, MINNEAPOLIS, MN, 55440, US

(21) Appl. No.: 10/075,321

(22) Filed: Feb. 14, 2002

(51) Int. Cl. A41D 13/08; A61F 9/00

(52) U.S. Cl. 2/16

(57) ABSTRACT

A chemical-resistant, user-wearable protective article. The article includes a first layer made of a polymeric material and a second layer made of a ceramic material. The second, ceramic material layer is coated onto the first layer. The first and second layers combined to form a flexible composite film that is shaped to be worn by a user as a barrier to chemicals. In one preferred embodiment, the second layer is aluminum oxide. Regardless, the film composite is preferably shaped as a glove, apron, etc., with the ceramic material coating providing a near perfect barrier to passage of most chemicals commonly employed in residential and industrial applications.

### BACKGROUND OF THE INVENTION

The present invention relates to a user-wearable protective article, for example gloves, apron, etc. More particularly, it relates to a user-wearable protective article formed with a ceramic-based composite film adapted to protect against adverse chemical contact, such as contracting contact dermatitis, when handling chemicals.

A wide variety of activities require persons to handle materials containing potentially hazardous chemicals. These activities range from in-home projects (e.g., applying stain to wood) to industrial endeavors such as the manufacture of certain products. Regardless, the chemicals involved may pose health risks if inhaled, ingested, brought into contact with the handler's skin, etc. Under these circumstances, then, a handler is well advised to employ appropriate measures to prevent possibly harmful interaction with the chemical(s) from occurring.

With respect to the prevention of skin contact, a multitude of protective apparel are available. For example, in most industrial applications where workers are required to handle potentially hazardous chemicals, the workers are commonly outfitted with rubber gloves, aprons, boots, etc. Alternatively, protective gear formed from other types of polymers and/or polymeric combinations have been developed. These materials have proven effective in minimizing the undesired contact between many chemicals and a handler's skin.

Unfortunately, certain potentially hazardous chemicals will readily pass through the available polymeric protective gear. For example, the preparation of certain food products entails use of a flavoring additive such as diacetyl (2, 3 Butanedione). Diacetyl and similar chemicals have been found to cause contact dermatitis in certain individuals who directly contact the chemical. In this regard, diacetyl will pass through rubber or other polymeric-based materials such that currently available protective gear offers minimal, if any, protection. A number of other chemicals present similar concerns as they inherently traverse through available protective gear material either immediately or over time with prolonged exposure.

Every effort should be made to provide chemical handlers with appropriate protective gear. Unfortunately, current safety apparel material does not provide a sufficient barrier to many commonly-used chemicals. Therefore, a need exists for improved protective gear formed of a material that provides a more complete barrier to chemicals that might otherwise cause an adverse reaction when directly contacting human skin.

### SUMMARY OF THE INVENTION

One aspect of the present invention relates to a chemical-resistant, user-wearable protective article. The article includes a first layer made of a polymeric material and a second layer made of a ceramic material. The second, ceramic material layer is coated onto the first layer. The first and second layers combined to form a flexible composite film that is shaped to be worn by a user as a barrier to chemicals. In one preferred embodiment, the second layer is aluminum oxide. Regardless, the film composite is preferably shaped as a glove, apron, etc., with the ceramic material coating providing a near perfect barrier to passage of most chemicals commonly employed in residential and industrial applications. For example, the ceramic material coating will prevent diacetyl from contacting the wearer's skin, thus protecting against potential contraction of contact dermatitis.

Another aspect of the present invention relates to a chemical-resistant protective glove including an outer layer, an immediate layer, and an inner layer. The outer layer is made of a polyester material. The intermediate layer is made of a ceramic material coated onto the outer layer. Finally, the inner layer is made of a heat sealable polymeric material that is bonded to the intermediate layer opposite the outer layer. The layers combine to form a flexible, composite film shaped as a glove and providing a user with a barrier to chemicals. In one preferred embodiment, the protective glove further comprises first and second sheets each formed of the composite film. In this regard, the inner layers of the first and second sheets are heat sealed to one another so as to define a perimeter of the glove.

Yet another aspect of the present invention relates to a method of manufacturing a chemical-resistant, user-wearable protective article. The method includes providing a flexible, composite film including a first layer made of a polymeric material coated with a second layer made of a ceramic material. The flexible, composite film is formed to a user-wearable shape and provides a barrier to chemicals. In one preferred embodiment, the method further includes bonding a third layer made of a heat-sealable polymeric material to the second layer opposite the first layer. In another preferred embodiment, the step of forming the flexible, composite film to a user-wearable shape includes defining a glove or an apron with the composite film.

### BRIEF DESCRIPTION OF THE DRAWINGS

FIG. 1 is a plan view of a user-wearable, protective article in accordance with the present invention;

FIG. 2 is a cross-sectional view of the article of FIG. 1 along the line 2-2;

FIG. 3 is a plan view of an alternative user-wearable, protective article in accordance with the present invention; and

FIG. 4 is a cross-sectional view of the article of FIG. 3.

### DESCRIPTION OF THE PREFERRED EMBODIMENTS

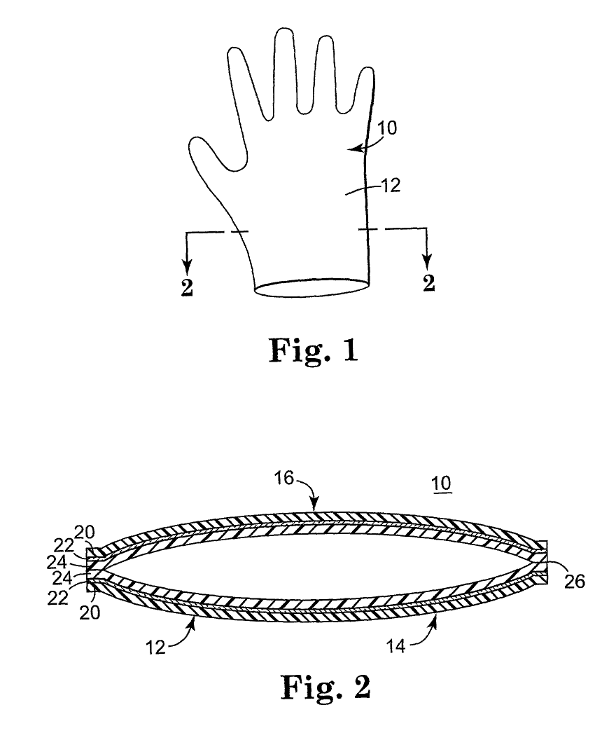

One preferred embodiment of a protective article 10 in accordance with the present invention is provided in FIG. 1. With the embodiment of FIG. 1, the protective article 10 is formed as a glove. Alternatively, the protective article 10 can assume a variety of other forms (e.g., apron, sleeves, etc.) as described in greater detail below.

Regardless of the exact form or shape, the protective article 10 is comprised of a flexible, composite film 12 as best shown in FIG. 2. With respect to the one preferred embodiment in which the protective article 10 is a glove, the composite film 12 is preferably formed as a first sheet 14 and a second sheet 16. Alternatively, where the protective article 10 assumes a different form or shape, only a single one of the sheets 14 or 16 need be provided. Regardless, in one preferred embodiment, the composite film 12 includes a first or outer layer 20, a second or intermediate layer 22, and a third or inner layer 24. Notably, for purposes of illustration, a thickness of the various layers 20-24 has been greatly exaggerated in FIG. 2. As described in greater detail below, the second layer 22 is a ceramic material that is coated onto the first layer 20. The third layer 24 is bonded to the second layer 22, resulting in the flexible, composite film 12.

The first layer 20 is a polymeric material support layer preferably in the form of a flexible film capable of receiving and maintaining a ceramic material coating (e.g., the second layer 22). Further, the first layer 20 material is preferably selected to provide strength and dimensional stability, as well as a chemical barrier property. That is, certain flexible film materials not only have the desired flexibility to provide support for the essential ceramic coating layer but also provide desirable chemical barrier properties themselves. With this in mind, the first layer 20 is preferably an oriented polyester film having a thickness of about 0.1-0.6 mil (2.5-15.2 microns). Thinner support layer thicknesses can be too fragile to resist tearing, especially during use, whereas excessively thick layers can be insufficiently flexible. Better results, in terms balancing cost and function are obtained when the film thickness is approximately 0.48 mil (12.2 microns). Alternatively, other polymeric materials such as nylon, Mylar, and polyolefins such as polyethylene, propylene and/or thicknesses are acceptable. If desired, the support film can itself be a laminate construction comprising multiple individual layers of various composition and structure.

As previously described, the second layer 22 is a ceramic material coated onto the first layer 20. In one preferred embodiment, the ceramic material is aluminum oxide (Al₂O₃), although other metal oxide materials such as silicon oxide (SiOₓ) are acceptable. Further, a laminate-like construction comprising multiple layers of differing ceramic materials can be employed if desired. As described in greater detail below, the ceramic material is coated onto the polymeric-based first layer 20 and provides an excellent barrier to most chemicals and/or odors.

Coating of the ceramic material second layer 22 onto the first layer 20 can be achieved in various fashions, but is preferably achieved through appropriate sputtering techniques in which micro-particles of the selected ceramic material are deposited onto the first layer 20. The term “sputtering” generally refers to a well-known technique involving deposition of gaseous material on to the support or first layer 20 in a vacuum chamber. The selected ceramic material(s) is given a charge to convert into a gaseous state and the vapor then deposits upon the substrate (i.e., the first layer 20) upon contact therewith. Alternatively, other available techniques, such as evaporation, gas plasma deposition, etc. are also available. Regardless, an appropriate composite film including aluminum oxide coated polyester is commercially available from Curwood of Oshkosh, Wis., under the tradename CURLAM® (Grade 8815-G).

The third layer 24 is also polymeric-based, but is selected to provide a heat sealable attribute. Further, where the first layer 20 is formed of a highly thin material (e.g., 0.48 mil polyester), the third layer 24 is selected to provide additional structural strength to the composite film 12. With this in mind, and in one preferred embodiment, the third layer 24 is a polyolefin such as polyethylene, polypropylene, Surlyn®, etc. More preferably, the third layer 24 is linear low density polyethylene (LLDPE). Regardless, the third layer 24 is bonded to the second layer 22, such as via a melt lamination process. By sandwiching the second layer 22 between the first and third layers 20, 24, the ceramic material comprising the second layer 22 is protected from scratching or other handling concerns. Further, at least one of the first and third layers 20, 24 are preferably characterized by a high tensile strength or low elongation, thereby preventing the composite film 12 from overtly stretching that might otherwise deteriorate the preferred uniform coating of the second layer 22.

With respect to the one preferred embodiment in which the protective article 10 is a glove, the inner or third layer 24 of each the first and second sheets 14, 16 are heat sealed to one another, resulting in a seam 26 as shown in FIG. 2. In this regard, each of the first and second sheets 14, 16 can be pre-cut to a desired shape (e.g., approximating a shape of a human hand) and then heat sealed to one another. Alternatively, the first and second sheets 14, 16 can be simultaneously heat cut and sealed to one another, resulting in the final shape. Regardless, the resulting article 10 provides a nearly complete barrier to many chemicals, especially those causing contact dermatitis in certain individuals. For example, in one preferred embodiment, the resulting article 10 is configured to be worn by a user who can then handle flavoring chemicals, such as diacetyl, with the article 10 preventing the diacetyl from contacting the handler's skin otherwise covered by the article 10. It will be understood that the protective article 10 serves as a barrier to a wide variety of other chemicals. Another advantage is that the protective article 10 is preferably impermeable to water, making such garments water proof. In still another variation, the present film 12 is employed as an intermediate or supplemental layer to conventional protective clothing to provide additional chemical protection.

The preferred glove article 10 is conveniently used as disposable items. For example, it is contemplated herein that such gloves could be added to home furniture refinishing kits to provide chemical hand protection against furniture stripping chemicals.

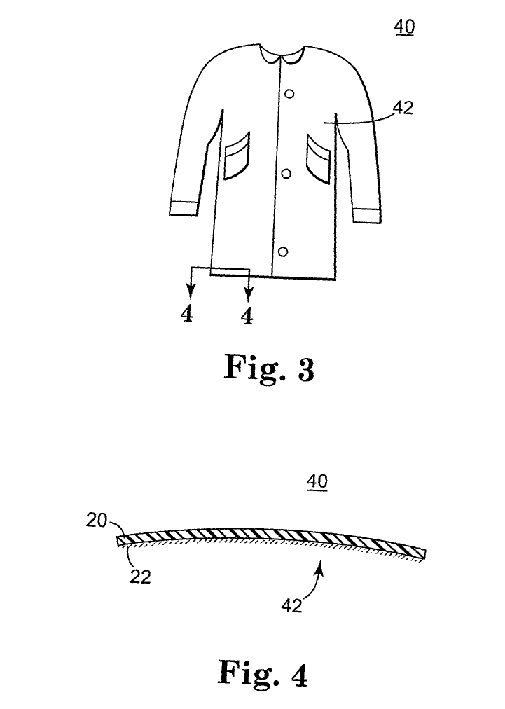

Although the protective article 10 has been preferably described as assuming the shape of a glove, other user-wearable formats are equally acceptable. For example, FIG. 3 depicts an alternative protective article 40 comprised of a flexible, composite material 42 shaped as a sleeved jacket or shirt. Other user-wearable articles can also be formed, such as aprons, sleeves, shoe coverings, etc. Additional examples include other clothing covers such as pants, coats, and head coverings such as hoods or shrouds.

In certain embodiments, the article(s) can be fabricated into whole body protective suits. Such whole body suits find use, for example, as protective garments for emergency response personnel responding to hazardous material emergencies such as chemical spills or biological hazards. For example, the suits can be used in situations wherein even gaseous contact with skin can cause serious or even fatal injury, e.g., responding to an industrial liquid chlorine gas spill during which the chlorine can vaporize, causing chemical burns when the gas or liquid contacts skin. In still other uses, the garments when combined with an appropriate sealed or protected breathing supply might provide protection against other gaseous agents that are toxic upon contact even at low concentration levels. Conventionally, such suits of protective clothing are single use items even though costing thousands of dollars per unit. Still another advantage of the present invention is that they are so inexpensive as to be disposable after a single use. When properly sealed, the whole body suits can provide protection against a wide variety of chemical and even biological agents when in liquid, solid or even gaseous form of mixtures thereof.

Still another advantage is that the present articles 10, 40 provide extreme barrier protection against highly permeable chemical agents such as methylene chloride or trichloroanasol which conventionally require glass for confinement yet nonetheless are highly flexible.

An additional feature of the alternative embodiment protective article 40 is best illustrated in FIG. 4. In particular, FIG. 4 illustrates the composite film 42 consisting of the first layer 20 and the second layer 22 as previously described. That is to say, as compared to FIG. 2, the film 42 of FIG. 4 eliminates the third, inner layer 24 (FIG. 2). In this regard, because the resulting article 40 (or at least portions thereof) is not sealed onto itself, the heat sealable third layer 24 is not necessary. It will be recognized, however, that even where the composite film 42 is not sealed onto itself, the third layer 24 as previously described can still be included for purposes of enhancing an overall structural strength of the film 42 and/or protecting the ceramic material-based second layer 22 from physical damage.

Composite films including polyester coated with a ceramic material, as well as ceramic-coated polyester materials laminated to relatively thick polyethylene, have been employed for health care packaging applications. In particular, health care products requiring sterilization have been packaged in ceramic-coated polyester film composites. Similarly, certain odorous chemicals are stored in bottles covered with ceramic-coated film composites for confining the odor. However, there has previously been no understanding or recognition of the chemical barrier attributes of ceramic coated polymeric film composites as it relates to user-wearable protective gear. Thus, the present invention represents a distinct advancement in the protective apparel industry, uniquely providing a chemical barrier material in the form of a wearable article that has sufficient flexibility so as to not impede normal activities (e.g., formed as a glove that a user can wear and still perform necessary hand manipulations).

An additional food processing-related application of the flexible, composite film of the present invention not otherwise previously recognized is in connection with packaging of food products requiring sanitization. In particular, the composite film 12 (FIG. 2) can be used to package a food item. The resulting packaged good article can then be subjected to super high pressure sanitizing operations without negatively impacting the quality of the contained food item. For example, it has been envisioned that subjecting a food item to extremely high pressures (on the order of 60,000-100,000 psi) will destroy the viability of certain, deleterious bacteria. A concern with this technique resides in the theory that the high-pressure environment may undesirably subject the food item to oxygen. However, the composite film 12 serves as a barrier to oxygen, thereby preventing the oxygen from interacting with the packaged food item.

Although the present invention has been described with reference to preferred embodiments, workers skilled in the art will recognize that changes can be made in form and detail without departing from the spirit and scope of the present invention.

What is claimed is:

1. A chemical-resistant, user-wearable protective article comprising:
a first layer made of a polymeric material; and
a second layer made of a ceramic material coated onto the first layer;
wherein the first and second layers combine to form a flexible film composite shaped to be worn by a user as a barrier to chemicals.

2. The protective article of claim 1, wherein the first layer is polyester.

3. The protective article of claim 1, wherein the second layer is a metal oxide.

4. The protective article of claim 3, wherein the second layer is aluminum oxide.

5. The protective article of claim 1, further comprising:
a third layer made of a heat sealable polymeric material bonded to the second layer opposite the first layer;
wherein the third layer defines an inner most layer and is configured to be sealable onto itself for forming the article.

6. The protective article of claim 5, wherein the third layer is polyethylene.

7. The protective article of claim 5, further comprising:
a first sheet formed of the composite film; and
a second sheet formed of the composite film;
wherein the third layer of the first and second sheets, respectively, are sealed to one another to define a perimeter of the protective article.

8. The protective article of claim 1, wherein the article is a glove.

9. The protective article of claim 1, wherein the article is an apron.

10. A chemical-resistant protective glove comprising:
an outer layer made of a polyester material;
an intermediate layer made of a ceramic material coated onto the outer layer; and
an inner layer made of a heat sealable polymeric material and bonded to the intermediate layer;
wherein the layers combine to form a flexible composite film shaped as a glove and providing a wearer with a barrier to chemicals.

11. The protective glove of claim 10, wherein the intermediate layer is aluminum oxide.

12. The protective glove of claim 10, further comprising:
a first sheet formed of the composite film; and
a second sheet formed of the composite film;
wherein the inner layers of the first and second sheets, respectively, are heat sealed to one another to define a perimeter of the glove.

13. A method of manufacturing a chemical-resistant, user-wearable protective article, the method comprising:
providing a flexible composite film including a first layer made of a polymeric material coated with a second layer made of a ceramic material; and
forming the composite film to a user-wearable shape providing a barrier to chemicals.

14. The method of claim 13, wherein providing a flexible film includes providing the first layer as a polyester material.

15. The method of claim 13, wherein providing a flexible composite film includes providing the second layer as aluminum oxide.

16. The method of claim 13, further comprising:
bonding a third layer made of a heat sealable polymeric material to the second layer opposite the first layer.

17. The method of claim 16, wherein the third layer is polyethylene.

18. The method of claim 16, wherein providing a flexible composite film includes providing a first sheet and a second sheet of the flexible composite film, and further wherein forming the flexible composite film includes heat sealing the third layer of the first sheet to the third layer of the second sheet.

19. The method of claim 13, wherein forming the flexible composite film includes defining a glove.

20. The method of claim 13, wherein forming the flexible composite film includes defining an apron.
